(19) United States
(12) Patent Application Publication
(10) Pub. No.: US 2012/0226213 A1
(43) Pub. Date: Sep. 6, 2012

(54) METHOD AND APPARATUS FOR PROVIDING PRESSURE ENHANCEMENT

(75) Inventor: Bernard Bar-Natan, Givatayim (IL)

(73) Assignee: FIRST CARE PRODUCTS LTD., Lod (IL)

(21) Appl. No.: 13/391,290

(22) PCT Filed: Aug. 19, 2010
(86) PCT No.: PCT/IL10/00678

§ 371 Date: May 7, 2012

(30) Foreign Application Priority Data
Aug. 20, 2009 (IL) 200508

(51) Int. Cl. A61F 13/00

(52) U.S. Cl. 602/53

(57) ABSTRACT

There is provided an apparatus for applying pressure to a wound, the apparatus comprising a main body portion having a top panel and two sides, the apparatus is adapted to be positioned on a dressing opposing a wound, such that the top panel is essentially parallel to a surface of a dressing. There is further provided a bandage comprising an elongated web, adapted to be folded upon itself, a dressing disposed on a wound surface of the web and a pressure enhancement member adapted to be directly or indirectly the dressing opposite the wound, wherein the pressure enhancement member comprises a main body portion having a top panel and two sides, the top panel is essentially parallel to the dressing.

### FIELD

The invention relates to bandages, such as pressure bandages, and devices for use therewith.

### BACKGROUND

A pressure (or compression) bandage may be in the form of an elongate strip (or “web”) of material (such as cloth, gauze or other material) for wrapping or binding an injured patient's body part, such as the patient's arm, leg, head or torso.

The purpose of bandages is to protect exposed wounds and, in relation to certain types of wounds, to prevent loss of blood. Bandages may be packaged to be carried and used by military personnel, police, ambulances, hikers and campers, for example, and may also be part of first-aid kits, for use in factories, offices, homes and on the road.

As described in commonly-owned U.S. Pat. No. 5,628,723 ('723 patent), bandages include a sterile dressing which includes one or more soft, absorbent portions, typically made of cotton, sandwiched between a (sterile) non-adherent pad and a structural layer. The pad is adapted to be placed on an open wound. The bandages also include a pressure enhancement member disposed on a non-wound side of the bandage, opposite the dressing. When the bandage is placed on the wounded body part, the pressure enhancement member should be guided to cover the wound such that a user (person applying the bandage, caregiver and/or victim/wounded person) can apply direct pressure to the wound. The subsequent wrappings of the web portion on the pressure enhancement member increases the direct pressure applied to the wound.

A variety of configurations for a pressure enhancement member, and using a bandage incorporating a pressure enhancement member are shown in the '723 patent.

For example, FIG. 3 of the '723 patent shows a bandage (100) which includes a sterile dressing (102), a web portion (104) and a pressure enhancement member (106). The pressure enhancement member comprises a base, and at least one wrapping element generally perpendicular to the base. The pressure enhancement member (106) may employ any of the frame members illustrated in FIGS. 1A-2H of the patent.

The pressure enhancement member (106) may be made of a flexible, resilient material, such as a structural plastic, and includes a bow-shaped portion (120) fixedly attached to a base (122) and generally perpendicular thereto.

Stainless steel and other metals and alloys, as well as tough generally inflexible plastics, are described as being suitable for the pressure enhancement member.

The frame member may have an internal space, and may include a gap in the continuity thereof, to enhance the ease with which the web is threaded into the internal space. During wrapping of the bandage onto a wounded body part, the web of the bandage may be passed though the gap in the frame of the pressure enhancement member into an interior space of the pressure enhancement member. The user should then go back, wrapping the bandage in the opposite direction. For the wrapping action, particularly for wrapping in the opposite direction, the user may take advantage of the elasticity of the web which facilitates the tight stretching of the web around the wounded body part. Wrapping the web in the opposite direction forces the pressure enhancement member down against the dressing and the pressure enhancement member thus applying pressure to the wounded area.

The '723 patent also shows and describes a hooking dowel (140) which is attached to an end (142) of the web portion (104). The hooking dowel (140) comprises a pair of opposing hooks (144). An end (146) of each hook (144) may be made of a material such as plastic or rubber. The hooking dowel (140) may include one or more prongs (147) to help prevent bunching and sliding of the web portion (104). In use, the hooking dowel (140) may be hooked into one of more folds (160) formed in the wrapping of web portion (104), thereby securing the bandage (100) around a wounded limb (150), and may facilitate the bandage (100) being used as a tourniquet.

In cases of emergency there is often an urgent need to stop a bleeding by applying weight on the wound and/or by applying a mechanical pressure. Only after a few seconds or minutes (generally when applying pressure becomes hard, the hand need to be released or attention should be drawn to other issues, such as attending other victims) the user wraps the bandage onto the wounded body part for the purpose of sustaining the applied pressure.

One of the difficulties with prior art pressure enhancement members relates to the limited area of the pressure enhancement member that actually applies a direct pressure on a wound. For example, as seen in FIG. 10A of the '723 patent, when wrapping the web, only the long edge of base (122) directly applies pressure against the wound. When a blood vessel (such as a vein or an artery) is cut, each of the two part of the cut vessel are often drawn back separating from each other (similar phenomena occurs when a stretched rubber band is cut). In this case applying pressure only along a relatively narrow line, such as the long edge of base (122) as shown in FIG. 10A of the '723 patent may “miss” one or even both of the blood vessel ends and, thus the bleeding will continue.

Another difficulty of prior art bandages relates to the grasping of the pressure enhancement member. As mentioned above, the user often stops the bleeding by applying weight (pressure) on the wound prior to wrapping the bandage. Existing structures of pressure enhancement members do not allow steady grasping by a user's hand. As a result the pressure applied against the wound may not the optimal pressure the user can apply.

Yet another difficulty of prior art bandages having pressure enhancement members relates to the difficulty in packing such bandages. Due to the bulkiness and stiffness of the pressure enhancement members the bandage packages are too big and occupy too much volume. This may cause difficulties particularly in military applications, where medics and other soldiers are required to carry a large amount of equipment and still maintain their mobility.

There is thus a need in the art for more efficient pressure enhancement members and more compact bandages that will facilitate urgent medical treatments.

### SUMMARY

This summary section of the patent application is intended to provide an overview of the subject matter disclosed herein, in a form lengthier than an “abstract”, and should not be construed as limiting the invention to any features described in this summary section.

It is a general object of the invention to provide improved bandages and improved techniques for using the bandages, such as for applying (direct) pressure to a wound.

According to some embodiments of the invention, generally, a device is provided, usable with a bandage, and the device may be sized and shaped to serve as a “pressure enhancement member” (or “handle”) for allowing a caregiver (user) to insert his hand into the device for applying pressure to a wound.

According to additional or alternative embodiments of the invention, generally, a device is provided usable with a bandage, the device may be adapted to serve as a “fold-retaining” device (or “holder”) for retaining a folded-up (or a rolled) bandage. The device may be adapted to serve as a packaging tool on the folded (or rolled) bandage and form a protective shell frame.

According to some embodiments of the invention, generally, a device is provided, usable with a bandage, the device may be adapted to serve as a “pressure enhancement member” (or “handle”) for allowing a user to insert his hand into the device for applying pressure to a wound and to serve as a “fold-retaining” device (or “holder”) for retaining a folded-up (or a rolled) bandage. Such configuration device may be referred to as a “combination holder/handle”.

According to an embodiment of the invention, there is provided an apparatus for applying pressure to a wound, the apparatus comprising: a main body portion having a top panel and two sides, the apparatus is adapted to be positioned on a dressing opposing a wound, such that the top panel is essentially parallel to a surface of a dressing. The apparatus of may be adapted to be directly or indirectly positioned on the dressing. The main body portion may generally be U-shaped in cross-section. The apparatus may be adapted to apply pressure to a wound, by a caregiver pressing against the wound on the top panel of the apparatus. The apparatus may be adapted to apply pressure to a wound, by a caregiver inserting his/her fingers to the apparatus and pressing against the wound on a bottom panel of the apparatus.

The top panel of the apparatus may generally be in the form of a rectangle. According to some embodiments, at least one of the two sides comprises an opening of sufficient size that a caregiver can insert fingers of a hand into the openings and apply pressure on the top panel by at least a portion of a palm of the hand.

According to some embodiments, the top panel comprises four corners: a first of the two sides comprises: a first elongated leg element extending from a first of the four corners of the top panel, a second elongated leg element extending from a second of the four corners of the top panel, a first elongated foot element extending between bottom ends of the first and the second leg elements and a first opening in the first of the two sides which is defined by the first and the second leg elements, the first foot element, and a first side edge of the top panel; a second of the two sides comprises: a third elongated leg element extending from a third of the four corners of the top panel, a fourth elongated leg element extending from a fourth of the four corners of the top panel, a second elongated foot element extending between bottom ends of the third and the fourth leg elements and a second opening in the second of the two sides which is defined by the third and the fourth leg elements, the second foot element, and a second side edge of the top panel. The top panel may be substantially rectangular. The foot elements may be substantially parallel with one another. The first, second, third and/or forth elongated leg elements may be substantially perpendicular to the top panel. The first elongated foot elements may extend substantially perpendicularly between bottom ends of the first and the second leg elements; and/or the second elongated foot element may extend substantially perpendicularly between bottom ends of the third and the fourth leg elements. The leg elements may extend substantially perpendicularly from an inner surface of the top panel, from the respective four thereof, for a portion of their length, then the leg elements angle inwardly. The first and the second leg elements may angle towards each other; and the third and the fourth leg elements may angle towards each other. The foot elements may be adapted to contact a bottom panel affixed to a bandage, opposite the dressing, wherein, in use, when a caregiver presses down on the top panel, the foot elements press down on the bottom panel, for distributing pressure to a larger area of the wound than would otherwise be applied by the foot elements alone. The apparatus may further comprise a bottom panel movably attached to the first foot element. The bottom panel may be integrally attached to the apparatus or movably attached to the first foot element by a living hinge.

According to some embodiments, in an open position the bottom panel extends up the first side of the apparatus, in an interior space between the two sides of the apparatus; and in a closed position, the bottom panel extends between the two foot elements at a bottom surface of the apparatus.

According to some embodiments, the apparatus, in use, when a caregiver presses down on the top panel, the foot elements press down on the bottom panel, for distributing pressure to a larger area of the wound than would otherwise be applied by the foot elements alone.

According to some embodiments, the top panel may generally be in the form of a rectangle having a left side edge, a right side edge, a front edge and a back edge, has a width dimension (W) which is a distance between the left side edge and right side edge, and has a length dimension (L) which is a distance between the front edge and the back edge; and may have the bottom panel may have a length dimension which is at least as great as the length dimension (L) of the apparatus, and may have a width dimension which is slightly less than the width (W) the apparatus.

According to some embodiments, the bottom portions of the third and the fourth leg elements may angle slightly inward, so that as the bottom panel swings open, the second foot element deflects outward, resiliently springing back into place so that the second foot is positioned on a top surface of the bottom panel. According to some embodiments, an inside surface of the second foot element is provided with a region of increased thickness.

According to some embodiments, a space between the two sides defines an interior space of the apparatus, and wherein the apparatus is sized and shaped so that a folded-up bandage may be at least partially inserted into the interior space of the apparatus.

According to some embodiments, the apparatus may be formed of a resilient material, and in use the two sides exert a resilient, retaining force on the folded-up bandage which is inserted into the interior space of the apparatus, and the folded-up bandage may be easily removed from the apparatus. The top panel may include a gap, wherein, in use, during wrapping of a bandage onto a wounded body part, a web of the bandage may be passed though the gap into an interior space which is defined between the two sides of the apparatus.

According to some embodiments, there is provided a bandage comprising an elongated web, adapted to be folded upon itself, a dressing disposed on a wound surface of the web and a pressure enhancement member adapted to be directly or indirectly the dressing opposite the wound, wherein the pressure enhancement member comprises a main body portion having a top panel and two sides, the top panel is essentially parallel to the dressing. The pressure enhancement member may be affixed to the elongated web, opposite the dressing. The main body portion may be generally U-shaped in cross-section. According to some embodiments, in use, the pressure enhancement member may be adapted to apply pressure to a wound, by a caregiver pressing against the wound on the top panel of the pressure enhancement member. According to alternative embodiments, in use, the pressure enhancement member may be adapted to apply pressure to a wound, by a caregiver pressing against the wound on a bottom panel of the pressure enhancement member. The top panel may generally be in the form of a rectangle. According to some embodiments, at least one of the two sides may include an opening of sufficient size that a caregiver can insert fingers of a hand into the openings and apply pressure on the top panel by at least a portion of a palm of the hand.

According to some embodiments, the top panel may include four corners; a first of the two sides may include: a first elongated leg element extending from a first of the four corners of the top panel, a second elongated leg element extending from a second of the four corners of the top panel, a first elongated foot element extending between bottom ends of the first and the second leg elements and a first opening in the first of the two sides which is defined by the first and the second leg elements, the first foot element, and a first side edge of the top panel; a second of the two sides may include: a third elongated leg element extending from a third of the four corners of the top panel; a fourth elongated leg element extending from a fourth of the four corners of the top panel; a second elongated foot element extending between bottom ends of the third and the fourth leg elements; and a second opening in the second of the two sides which is defined by the third and the fourth leg elements, the second foot element, and a second side edge of the top panel. According to some embodiments, the top panel may be substantially rectangular in form. According to some embodiments, the foot elements may be substantially parallel with one another. According to some embodiments, the first, second, third and/or forth elongated leg elements may be substantially perpendicular to the top panel. According to some embodiments, the first elongated foot elements may extend substantially perpendicularly between bottom ends of the first and the second leg elements; and/or wherein the second elongated foot element extends substantially perpendicularly between bottom ends of the third and the fourth leg elements. According to some embodiments, the leg elements may extend substantially perpendicularly from an inner surface of the top panel, from the respective four thereof, for a portion of their length, then the leg elements angle inwardly. According to some embodiments, the first and the second leg elements may angle towards each other; and the third and the fourth leg elements may angle towards each other. The foot elements may be adapted to contact a bottom panel affixed to the bandage, opposite a dressing, wherein, in use, when a caregiver presses down on the top panel, the foot elements press down on the bottom panel, for distributing pressure to a larger area of the wound than would otherwise be applied by the foot elements alone. According to some embodiments, the bandage may further include a bottom panel movably attached to the first foot element. The bottom panel may be movably attached to the first foot element by a living hinge. According to some embodiments, in an open position the bottom panel extends up the first side of the pressure enhancement member, in an interior space between the two sides of the pressure enhancement member and in a closed position, the bottom panel extends between the two foot elements at a bottom surface of the pressure enhancement member. According to some embodiments, in use, when a caregiver presses down on the top panel, the foot elements may press down on the bottom panel, for distributing pressure to a larger area of the wound than would otherwise be applied by the foot elements alone. According to some embodiments, the top panel may generally be in the form of a rectangle having a left side edge, a right side edge, a front edge and a back edge, has a width dimension (W) which is a distance between the left side edge and right side edge, and has a length dimension (L) which is a distance between the front edge and the back edge; and the bottom panel has a length dimension which is at least as great as the length dimension (L) of the pressure enhancement member, and has a width dimension which is slightly less than the width (W) the pressure enhancement member. According to some embodiments, bottom portions of the third and the fourth leg elements may angle slightly inward, so that as the bottom panel swings open, the second foot element deflects outward, resiliently springing back into place so that the second foot is positioned on a top surface of the bottom panel. According to some embodiments, an inside surface of the second foot element may be provided with a region of increased thickness. According to some embodiments, a space between the two sides may define an interior space of the pressure enhancement member, and wherein the pressure enhancement member is sized and shaped so that a folded-up bandage may be at least partially inserted into the interior space of the pressure enhancement member. The pressure enhancement member is may be formed of a resilient material, and in use the two sides exert a resilient, retaining force on the folded-up bandage which is inserted into the interior space of the pressure enhancement member, and the folded-up bandage may be easily removed from the pressure enhancement member. The top panel may include a gap, wherein, in use, during wrapping of the bandage onto a wounded body part, a web of the bandage may be passed though the gap into an interior space which is defined between the two sides of the pressure enhancement member.

### BRIEF DESCRIPTION OF FIGURES

Examples illustrative of embodiments of the invention are described below with reference to figures attached hereto. In the figures, identical structures, elements or parts that appear in more than one figure are generally labeled with a same numeral in all the figures in which they appear. Dimensions of components and features shown in the figures are generally chosen for convenience and clarity of presentation and are not necessarily shown to scale. The figures (FIGS.) are listed below.

FIG. 1A schematically shows, in cross-sectional view, a folded bandage.

FIG. 1B schematically shows, in cross-sectional view, the bandage of FIG. 1, unfolded.

FIG. 2 schematically shows, in perspective view; a device capable of functioning as a “holder” and a “handle”, according to an embodiment of the invention.

FIG. 2A schematically shows, in perspective view, the device of FIG. 2, functioning as a “holder”.

FIG. 2B schematically shows, in perspective view, the device of FIG. 2, functioning as a “handle”.

FIG. 3 schematically shows, in perspective view, a device capable of functioning as a “holder” and a “handle”, according to an embodiment of the invention.

FIG. 3A schematically shows, in cross-sectional view, the device of FIG. 3, functioning as a “holder”.

FIG. 3B schematically shows, in cross-sectional view, the device of FIG. 3, functioning as a “handle”.

FIG. 3C schematically shows, in cross-sectional view, the device of FIG. 3, functioning as a “handle”.

FIG. 3D schematically shows, in cross-sectional view, the device of FIG. 3, functioning as a “handle”.

FIG. 4 schematically shows, in cross-sectional view, a bandage in combination with the device of FIG. 3, with the device serving as a “holder”, according to an embodiment of the invention.

FIG. 4A schematically shows, in cross-sectional view, the bandage and device of FIG. 4, with a user (caregiver) starting to remove the bandage from the device.

FIG. 4B schematically shows, in cross-sectional view, the bandage and device of FIG. 4, with the device in place to serve as a pressure enhancement member, or “handle”.

FIG. 4C schematically shows, in perspective view, the bandage and device of FIG. 4, with the device in place to serve as a pressure enhancement member, or “handle”.

FIG. 4D schematically shows, in perspective view, the bandage and device of FIG. 4, with the device in place to serve as a pressure enhancement member, or “handle”.

FIG. 4E schematically shows, in perspective view, the bandage and device of FIG. 4, with the device in place to serve as a pressure enhancement member, or “handle”.

FIG. 5 schematically shows, in perspective view, an unfolded bandage with the combination holder/handle device of FIG. 4, in use.

### DETAILED DESCRIPTION OF EMBODIMENTS

In the following description, various aspects of techniques for applying pressure to a wound and/or packaging bandages will be described. For the purpose of explanation, specific configurations and details are set forth in order to provide a thorough understanding of the techniques. However, it will also be apparent to one skilled in the art that the techniques may be practiced without specific details being presented herein. Furthermore, well-known features may be omitted or simplified in order not to obscure the description(s) of the techniques.

After manufacture, the bandage (either with or without integral dressing) may be rolled up and packaged for sale and distribution. An alternative to rolling up the bandage is to fold it. Folding a material once (n=1) will result in there being two (n+1) “panels”, each of which may (or may not) be the same size as each other. The resulting panels are generally parallel with one another, and substantially abutting one another (like an accordion or a concertina).

A material may be folded with “zigzag” folds, will have two or more (n>=2) parallel folds, each folding the material in opposite directions, resulting in a number (n+1) of panels, each of which may (or may not) be the same size as each other. The resulting panels are generally parallel with one another, and substantially abutting one another. Seen from above, zig-zag folds resemble a Z, W or M. Zigzag folds are also known as “accordion folds” or “z folds”. A typical pressure bandage may be zig-zag folded, with several (such as n=5-10) folds, resulting in several (n+1) panels.

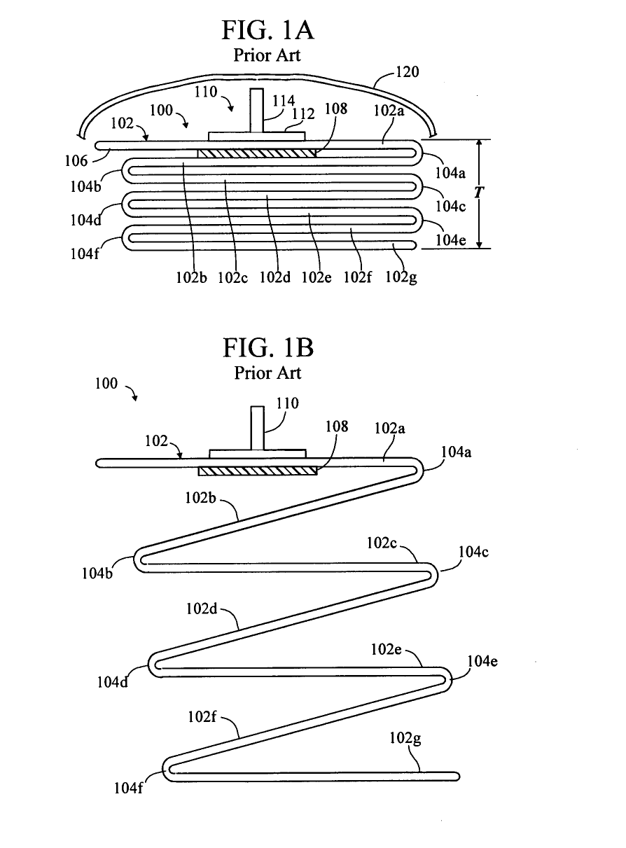

FIG. 1A illustrates a bandage 100, generally comprising an elongate web 102. The web 102 is folded upon itself, such as zig-zag folded, with six (n=6) folds 104a-104f, resulting in seven (n+1) panels 102a-102g. Of course, n can represent any number (such as 3, 4, 5, 6, 7, 8 . . . n), 6 being just an example. The bandage 100 may be referred to as an “emergency bandage”.

The panel 102a is an “end panel”. A dressing 108 may be disposed on a surface of the end panel 102a. A pressure enhancement member 110, such as described in commonly-owned U.S. Pat. No. 5,628,723, may be disposed on a surface of the end panel 102a opposite the dressing 108.

It may be noted that the end panel 102a has been drawn slightly longer than the rest of panels 102b-g. This is indicative of an “additional” web portion 106, which typically extends about 5 cm from the dressing 108 and which may be grasped by a user when applying the bandage 100 on a wound (not shown).

In FIG. 1A, the bandage 100 is shown in its folded configuration, neatly folded, with the panels 102a-102g generally parallel with one another, and substantially abutting one another. For example, the panel 102a abuts the panel 102b, the panel 102b abuts the panels 102a and 102c, the panel 102c abuts the panels 102b and 102d, the panel 102d abuts the panels 102c and 102e, the panel 102e abuts the panels 102c and 102f, and the panel 102f abuts the panels 102e and 102g.

Typical dimensions for a pressure bandage (namely, the web) may be 10-15 cm wide, and at least one meter long. Typical dimensions for the dressing 108 may be 10-15 cm square. The web material may be 0.25-1.5 mm thick. When folded-up, the bandage 100 has an overall thickness “T”, exclusive of the pressure enhancement member 110. The thickness “T” may, for example, be approximately 3 cm (30 mm). In this example, the overall thickness “T” is approximately equal to seven times the thickness (approximately 4 mm) of each panel (102a-102f), plus the thickness of the dressing 108, taking into consideration that the web and dressing are formed of “soft” materials (such as cotton, gauze, elasticized fabric, and the like) which may be compressed when packaged.

As illustrated in FIG. 1A, the pressure enhancement member 110 comprises a base portion 112 which lays essentially flat on the surface of the web 102 of the bandage 100, and a portion 114 which extends perpendicularly from the base portion 112, hence from the web 102 of the bandage 100, and therefore will cause the “bandage with pressure enhancement member” to occupy a significantly larger space (volume) within a package (bag) 120 than the bandage 100 by itself, as well as creating a very irregular shape for the packaged “bandage with pressure enhancement member”. The bag 120 is shown only partially, in FIG. 1A.

When the bandage 100 is removed from its package (not shown), the user (person applying the bandage) may hold (grasp) it in their hand(s), thereby maintaining the bandage 100 in its zig-zag folded configuration. But in the likely stressful and chaotic conditions surrounding use of the bandage, the user may lose their grip on or otherwise fumble the folded bandage 100, resulting for example in the bandage 100 unfolding and dropping or extending to the ground. This is generally undesirable since not only can the bandage become contaminated, but it can also become unwieldy, or snagged on something. This “unfolded” condition of the bandage 100 is illustrated in FIG. 1B. It should be understood that, when unfolded, there may not be these small (such as 15 degree) angles between successive panels, but rather the entire bandage 100 would tend to straighten out until it hit the ground.

According to some embodiments, the web may not be folded in a zigzag manner. Rather, the web portions may be rolled up, for example in a flattened spiral manner. This can also be considered to be a type of “folding”.

There will now be described embodiments of method and apparatus for maintaining the panels of a folded, such as zig-zag folded bandage abutting one another, and applying pressure to a wound while applying the bandage thereto. The devices described herein may be referred to herein as “fold-retaining” devices and/or “pressure enhancement members”, depending generally on whether the device/member is being used to retain the bandage in its folded configuration, to apply pressure to a wound or both.

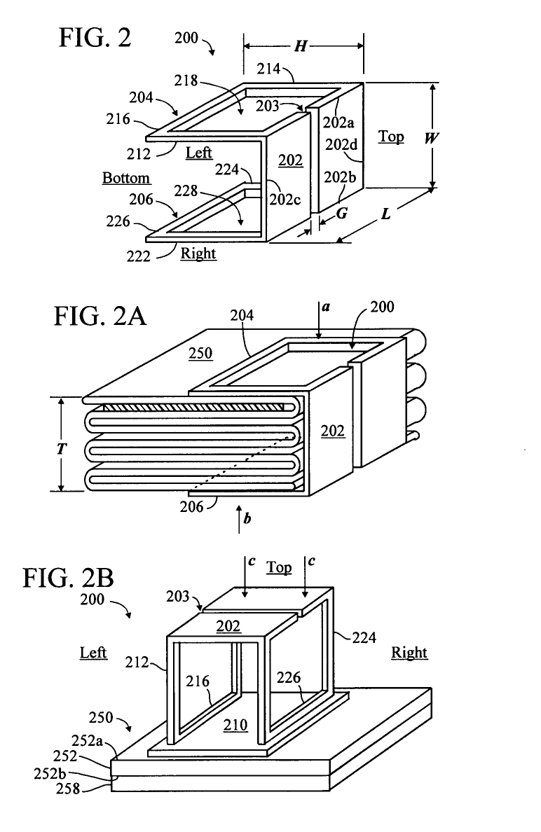

FIGS. 2, 2A and 2B illustrate an embodiment of a device 200 which may be used as (and is sized and shaped to function as) a “holder” for retaining a folded-up bandage in its folded configuration, and as a pressure enhancement member (or “handle”) for applying pressure to a wound.

A main body portion of the device 200 may be generally U-shaped in cross-section, having a top panel 202 generally in the form of a rectangle having a width “W” dimension and a length dimension “H”. The width dimension “W” is a distance between a left side edge 202a and a right side edge 202b of the top panel 202, and the length dimension is a distance between a front edge 202c and a back edge 202d of the top panel 202. The left side edge 204a is opposite the right side edge 204b, and the front side edge 204c is opposite the back side edge 204d. The top panel 202 has four corners, as follows:
a left-front (LF) corner of the top panel 202 is formed at the intersection of the left edge 202a and the front edge 202c of the top panel 202;
a left-back (LB) corner of the top panel 202 is formed at the intersection of the left edge 202a and the back edge 202d of the top panel 202;
a right-front (RF) corner of the top panel 202 is formed at the intersection of the right edge 202b and the back edge 202d of the top panel 202; and
a right-back (RB) corner of the top panel 202 is formed at the intersection of the right edge 202b and the back edge 202d of the top panel 202.

A first (left) side 204 of the device 200 comprises:
an elongated “leg” element 212 extending from the left-front (LF) corner of the top panel 202, substantially perpendicular to the top panel 202;
an elongated “leg” element 214 extending from the left-back (LB) corner of the top panel 202, substantially perpendicular to the top panel 202;
an elongated “foot” element 216 extending substantially perpendicularly between bottom ends of the leg elements 212 and 214.

An opening 218 in the left side 204 of the device 200 is defined by the leg elements 212 and 214, the foot element 216 and the left side edge 202a of the top panel 202.

A second (right) side 206 of the device 200 comprises:
an elongated “leg” element 222 extending from the right-front (RF) corner of the top panel 202, substantially perpendicular to the top panel 202;
an elongated “leg” element 224 extending from a right-back (RB) corner of the top panel 202, substantially perpendicular to the top panel 202;
an elongated “foot” element 226 extending substantially perpendicularly between bottom ends of the leg elements 222 and 224.

An opening 228 in the right side 204 of the device 200 is defined by the leg elements 222 and 224, the foot element 226 and the right side edge 202b of the top panel 202.

All four leg elements 212, 214, 222 and 224 may be substantially parallel with one another, and the two foot elements 216 and 226 may be substantially parallel with one another.

The leg elements 212, 214, 222, 224 may each have a length dimension which is substantially equal to the height “H” of the device 200.

The foot elements 216 and 226 may each have a length dimension which is substantially equal to the length “L” of the device 200.

A space between the two sides 204 and 206 may be referred to as the “interior space” of the device 200, and may have a width dimension which is the dimension “W” (less the thickness of the leg elements 212, 214, 222, 224). The openings 218 and 228 extend from an exterior of the device 200 to the interior space of the device 200.

A gap 203 may be situated (disposed) approximately halfway along the length “L” of the top panel 202, entirely across the width “W” of the top panel 202, and into the left side opening 218 and the right side opening 228. The gap 203 may have a width dimension “G”. The gap 203 leads into the interior space of the device 200.

In geometric terms, the device 200 is a six-sided rectangular prism, having for example:
a top surface which is defined by the top panel 202. The top surface is continuous (or closed), except for the gap 203.
a bottom surface, opposite the top surface, which is defined by the foot elements 216 and 226. The bottom surface is substantially open, except for the foot elements 216 and 226.
a left side 204 which is defined by the two legs elements 312 and 214, and the foot element 216. The left side 204 is substantially open.
a right side 206, opposite the left side, which is defined by the two leg elements 222 and 224, and the foot element 226. The right side 206 is substantially open.
a front end which is defined by the leg elements 212 and 222, and the front edge 202c of the top panel 202. The front end is substantially open.
a back end which is defined by the leg elements 214 and 224, and the back edge 202d of the top panel 202. The back end is substantially open.

Although not shown, an alternative to a substantially “closed” top panel 202 would be to form the top panel 202 in a manner similar to the side panels 204 and 206, but with two openings separated by a gap.

FIG. 2A illustrates the device 200 functioning as a “holder”, such as for packaging a folded-up emergency bandage. A folded-up bandage 250 may be at least partially inserted into (disposed within) the interior space of the device 200. The folded-up bandage 250 may be similar to the bandage 100. The folded-up bandage 250 has a thickness “T” which is approximately equal to (or slightly greater than) the width of the interior space. Hence, the folded-up bandage 250 may be retained in its folded-up configuration by the device 200.

The device may be made of a resilient material, such as plastic, so that the left and right sides 204 and 206 can exert a resilient, retaining force on the folded-up bandage 250, as indicated by the arrows “a” and “b”. In use, the folded-up bandage 250 is easily removed from the holder 200.

The device 200, as holder, is sized and shaped, to fit at least partially around the folded bandage 250 in a manner that does not substantially increase the total volume of a package (not shown, compare 120) for the bandage 250, and does not substantially detract from the otherwise regular, rectangular prismatic shape of the packaged bandage, so that it can still be space-efficiently packed with other similar (such as identical) items.
The device 200 may be made of a resilient plastic material, such as polycarbonate, polypropylene, polyethylene with nylon fibers or any other material, such as by injection molding.

A caregiver (user) may take-out the folded-up bandage 250 from holder 200 (or, conversely, remove the holder from the bandage), unfold it, and apply it to a patient's wound. In the case of hemorrhaging, it is recommended to provide direct pressure to the wound to assist in the homeostasis process.

FIG. 2B illustrates the device 200 in use as a pressure enhancement member, or “handle”. The bandage 250 comprises an elongated web 252 having two surfaces 252a and 252b. The surface 252b is designated the “wound surface”, and a dressing 258 may be integrated on the wound surface 252b. The device 200 may be positioned with its foot elements 216 and 226 disposed against the web surface 252a, opposite the dressing 258. The user can apply pressure, via the device 200, to the wound.

In FIG. 2B, rather than each foot element 216 and 226 applying pressure, individually, to the wound, in this embodiment, a panel 210 may be located on the web surface 252a, opposite the dressing 258, and affixed to the bandage 250. The panel 210 may have a length dimension which is at least as great as the length dimension “L” of the device 200, and may have a width dimension which is at least as great as the width dimension “W” of the device 200. In use, when a caregiver presses down on the device 200, as in indicated by the arrows “c”, the foot elements 216 and 226 (which may be adapted to hook up to panel 210, for example by clip(s) or a groove in panel 210 adapted to capture foot elements 216 and 226, not shown) press down on the panel 210, and the pressure is distributed from the panel 210 to a larger area of the wound than would otherwise be applied by foot elements 216 and 218 alone. The panel 210 may be referred to as a “pressure-spreading” element.

Detents, pins or the like (not shown) may be provided on the panel 210 and/or the foot elements 216 and 226 to help properly locate the foot elements 216 and 226 on the panel 210.

A feature of this embodiment is that the device 200 is configured (sized and shaped) so that a caregiver can insert his hand (fingers of his hand) via one of the openings (318 or 228), into the interior space of the device, and out the other opening (328 or 218), securely grasping the device 200, while applying direct pressure to the wound.

After removing the folded-up bandage 250 from the device 200, the device 200 may be used as a pressure-enhancement member for applying pressure, via the bandage 250, to a wound. Notice that the gap 203 is comparable to the gap in the frame member of the pressure enhancement member of the '723 patent. And, in a manner comparable to what was described therein:

During wrapping of the bandage onto a wounded body part, the web of the bandage may be passed though the gap in the frame of the pressure enhancement member into an interior space of the pressure enhancement member. The user then goes back, wrapping the bandage in the opposite direction. Wrapping the web in the opposite direction forces the pressure device 200 down (tilting it essentially 90° to the right or to the left) against the dressing such that the pressure enhancement member applies pressure to the wounded area. Panel 210 now extends generally perpendicularly away from the wound area. When device 200 is forced to the left, the long edge of panel 210 in proximity to “foot” element 216) and left side edge 202a apply pressure on the wound while “leg” elements 212 and 214 lie essentially flat on surfaces 252a (indirectly on dressing 258). The user may continue wrapping the bandage, such that the web covers the tilted device 200, thus applying more pressure on the long edge of panel 210 (“foot” element 216 or both, depending on the geometry) and left side edge 202a, which apply pressure on the wound. As opposed to prior art bandages, pressure is being applied essentially along two lines, namely the long edge of panel 210 (“foot” element 216 or both, depending on the geometry) and left side edge 202a. This may be advantageous to simultaneously stop a bleeding from two separated ends of a torn blood vessel (such as a vein or an artery).

A “problem” with the device 200 is that upon removing the folded-up bandage 250 from the device 200, the caregiver could drop the device 200, and could then be distracted by retrieving it. Therefore, in the next embodiment, a device 300 is described which includes an additional pressure-spreading element (compare 210) which is attached to the device, as well as being affixed to the bandage. As will be seen, upon removing the bandage from the device (as “holder”) the device will reposition itself to be useful as a pressure enhancement member (“handle”), appropriately positioned opposite the dressing.

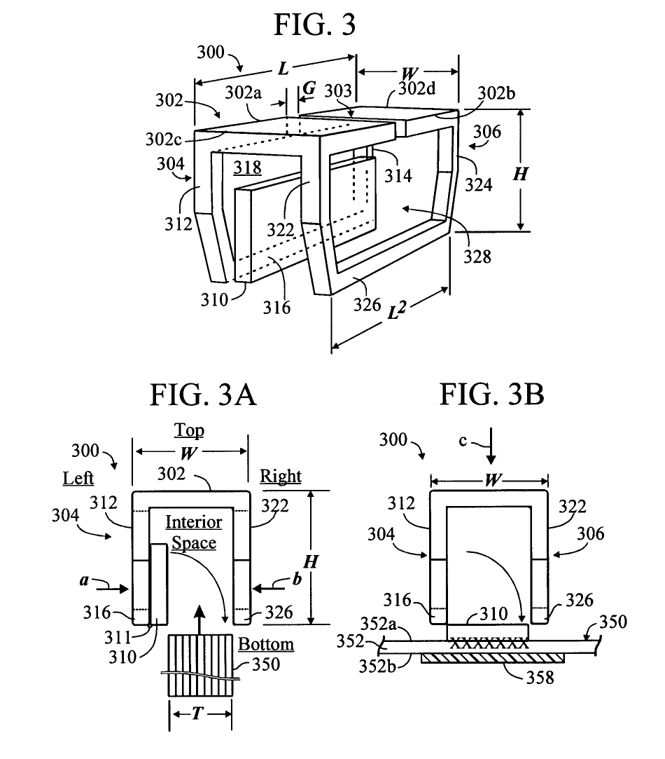

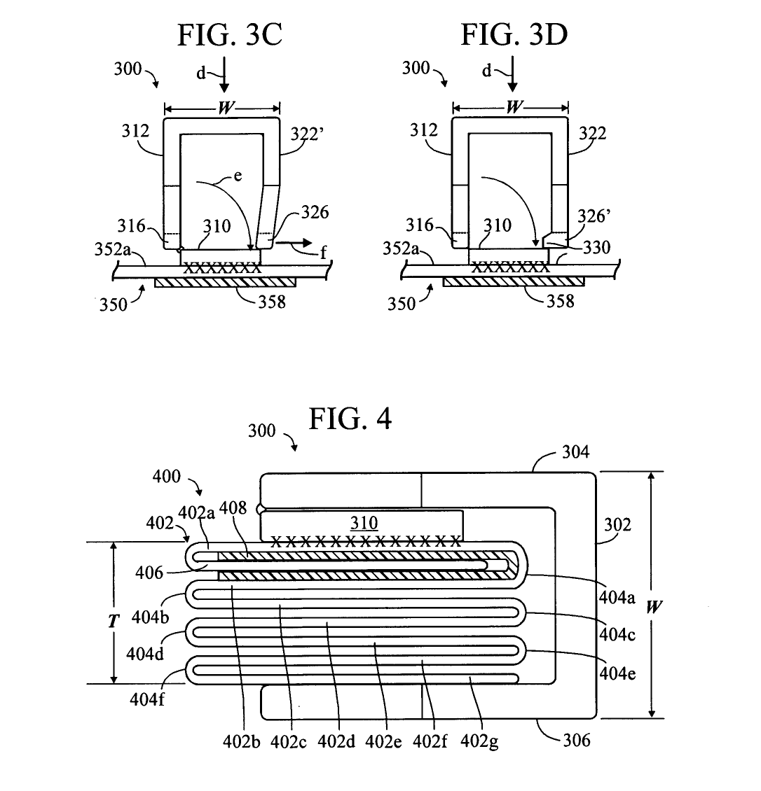

FIGS. 3, 3A and 3B illustrate another embodiment of a device 300 which may be used as a “holder” for retaining a folded-up bandage in its folded configuration, and as a pressure enhancement member (or “handle”) for applying pressure to a wound.

A feature of this embodiment is that the handle is configured (sized and shaped) so that a caregiver can insert his hand through the device, securely grasping it, while applying direct pressure to the wound.

Another feature of this embodiment is that the device may be permanently attached to a web of a bandage, such as on an opposite side of the web from a dressing which also may be permanently attached to the web of the bandage. Alternatively, the device may be releasably attached to a web of a bandage, such as using a hook and loop system.

A main body portion of the device 300 may be generally U-shaped in cross-section, having a top panel 302 (compare 202) generally in the form of a rectangle having a width “W” dimension and a length dimension “L”. The width dimension “W” is a distance between a left side edge 302a and a right side edge 302b of the top panel 302, and the length dimension “L” is a distance between a front edge 302c and a back edge 302d of the top panel 302. The left side edge 304a is opposite the right side edge 304b, and the front side edge 304c is opposite the back side edge 304d. The top panel 302 has four corners, as follows:
a left-front (LF) corner of the top panel 302 is formed at the intersection of the left edge 302a and the front edge 302c of the top panel 302;
a left-back (LB) corner of the top panel 302 is formed at the intersection of the left edge 302a and the back edge 302d of the top panel 302;
a right-front (RF) corner of the top panel 302 is formed at the intersection of the right edge 302b and the back edge 302d of the top panel 302; and
a right-back (RB) corner of the top panel 302 is formed at the intersection of the right edge 302b and the back edge 302d of the top panel 302.

A first (left) side 304 (compare 204) of the device comprises:
an elongated “leg” element 312 extending from the left-front (LF) corner of the top panel 302, substantially perpendicularly to the top panel 302;
an elongated “leg” element 314 extending from a left-back (LB) corner of the top panel 202, substantially perpendicular to the top panel 302; and
an elongated “foot” element 316 extending substantially perpendicularly between bottom ends of the leg elements 312 and 314.

An opening 318 in the left side 304 of the device 300 is defined by the leg elements 312 and 314, the foot element 316 and the left side edge 302a of the top panel 302.

A second (right) side 306 (compare 206) of the device comprises:
an elongated “leg” element 322 extending from a right-front (RF) corner of the top panel 302, substantially perpendicular to the top panel 302;
an elongated “leg” element 324 extending from a right-back (RB) corner of the top panel 302, substantially perpendicular to the top panel 302;
an elongated “foot” element 326 extending substantially perpendicularly between bottom ends of the leg elements 324 and 326.

An opening 328 in the left side 304 of the device 300 is defined by the leg elements 322 and 324, the foot element 326 and the right side edge 302b of the top panel 302.

All four leg elements 312, 314, 322 and 324 may be generally parallel with one another, and the two foot elements 316 and 326 may be substantially parallel with one another.

The leg elements 312, 314, 322, 324 may each have a length dimension which is substantially equal to the height “H” of the device 300.

The foot elements 316 and 326 may each have a length dimension which is substantially equal to the length “L” of the device 200.

A space between the two sides 304 and 306 may be referred to as the “interior space” of the device 300, and has a width dimension which is the dimension “W” (less the thickness of the leg elements 312, 314, 322, 324). The openings 318 and 328 extend from an exterior of the device 300 to the interior space of the device 300.

A gap 303 may be situated approximately halfway along the length “L” of the top panel 302, entirely across the entire width “W” of the top panel 302, and into the left side opening 318 and the right side opening 328. The gap 303 may have a width dimension “G”. The gap 303 leads into the interior space of the device 300.

In geometric terms, the device 300 is a six-sided prism, having for example:
a top surface which is defined by the top panel 302. The top surface is continuous (or closed), except for the gap 303.
a bottom surface, opposite the top surface, which is defined by the feet 316 and 326. The bottom surface is substantially open, except for the feet 316 and 326.
a left side 304 which is defined by the two legs 312 and 314, and the foot 316. The left side 304 is substantially open.
a right side 306, opposite the left side, which is defined by the two legs 322 and 324, and the foot 326. The right side 306 is substantially open.
a front end which is defined by the legs 312 and 322, and the front edge 302c of the top panel 302. The front end is substantially open.
a back end which is defined by the legs 314 and 324, and the back edge 302d of the top panel 302. The back end is substantially open.

Up to here, the description of the device 300 is substantially the same as the description of the device 200. Some differences will now be discussed, generally involving the legs being angled (rather than straight), and a pressure-spreading element which is hingedly attached to the device, as well as being affixed to the bandage. And, as mentioned above, the top panel may be formed with two openings separated by a gap.

Rather than the legs 312, 314, 324, 326 being straight, they may be bent (bowed, rounded or angled), as follows. The legs 312 and 314, and the legs 322 and 324 extend substantially perpendicularly from an inner (towards the “interior space”) surface of the top surface panel 302, from the respective four corners LF and LB, RF and RB thereof, for a portion of their length. Then, the legs 312, 314, 322 and 324 may angle inwardly, as follows.
At a midpoint (approximately halfway) along its length, the leg 312 angles towards the leg 314. The leg 312 does not angle towards the leg 322, but remains substantially parallel therewith.
At a midpoint (approximately halfway) along its length, the leg 314 angles towards the leg 312. The leg 314 does not angle towards the leg 324, but remains substantially parallel therewith.
At a midpoint (approximately halfway) along its length, the leg 322 angles towards the leg 324. The leg 316 does not angle towards the leg 312, but remains substantially parallel therewith.
At a midpoint (approximately halfway) along its length, the leg 324 angles towards the leg 322. The leg 324 does not angle towards the leg 314, but remains substantially parallel therewith.

Since bottom portions of the legs 312 and 322 may angle towards bottom portions of the legs 314 and 324, respectively, a length dimension “L2” at the bottom of the device 300 may be less than the length dimension “L” at the top of the device.

A generally rectangular panel 310 (compare 210) is disposed at the bottom end of the device 300, and may be movable between two positions—an “open” position (see also FIG. 3A) and a “closed” position (see also FIG. 3B). In its open position, the panel 310 may extend up and abut the left side surface 304 of the device 300 (in the interior space). In its closed position, the panel 310 extends between the two foot elements 316 and 326, and may be substantially coincident with the bottom surface of the device 300.

The panel 310, which may be referred to as a “bottom” panel or a “bottom” surface (although, in the open position it is along the side of the device) may be movably attached (hinged) to the bottom left foot 316, such as with a “living hinge”. (A living hinge is a thin flexible hinge (flexure bearing) made from plastic (rather than cloth, leather, or some other substance) that joins two rigid plastic parts together, allowing them to bend along the line of the hinge. It is typically manufactured in an injection molding operation that creates all three parts at one time as a single part, and if correctly designed and constructed, it can remain functional over the life of the part. Polyethylene and polypropylene are considered to be the best resins for living hinges, due to their excellent fatigue resistance.) The panel 310 may have a length dimension which is at least as great as the length “L” of the device 300, and a width dimension which is slightly less than the width “W” of the device 300.

FIG. 3A is a view looking into the front end of the device 300, with the panel 310 in its “open” position, extending in the interior space up the left side surface 304 of the device 300. As illustrated, there is a hinge 311, such as a living hinge joining a left side edge of the panel 310 with the left side foot 316 (hidden behind the leg 312).

The view of FIG. 3A is similar to the view of FIGS. 2A, but rotated 90-degrees, and illustrates the device 300 functioning as a “holder”, such as for packaging a folded-up emergency bandage. A folded-up bandage 350, shown generically in this view, may be at least partially inserted into the opening between the feet 316 and 326, in the bottom end of the device 300, as indicated by the upward-pointing arrow extending from the bandage 350 into the interior space of the device 300, and may be releasably retained therein (in the sense that the bandage can easily be taken out of the device) by resilient pressure exerted by the legs 312 and 322 (and 314, 324, not visible), as indicated by the sidewards-pointing arrows “a” and “b”. The folded-up bandage 250 may be similar to the bandage 100. The folded-up bandage 350 has a thickness “T” which is approximately equal to (or slightly greater than) the width of the interior space. Hence, the folded-up bandage 350 may be retained in its folded-up configuration by the device 300.

The view of FIG. 3B is similar to the view of FIG. 2B, and illustrates the device 300 in use as a pressure enhancement member, or “handle”. The bandage 350 comprises an elongated web 352 having two surfaces 352a and 352b. The surface 352b is designated the “wound surface”, and a dressing 358 may be integrated on the wound surface 352b. The device 300 may now be positioned with its foot elements 316 and 326 disposed towards the web surface 352a, opposite the dressing 358, and the caregiver can apply pressure, via the device 200, to the wound.

In a manner similar to the previous embodiment (300), in this embodiment 300 the foot elements 316 and 326 may apply pressure via the panel 310 to the wound. The panel 310 may be located on the web surface 352a, opposite the dressing 358, and affixed to the bandage 350. (The x's indicate that the panel 310 is attached to the web surface 352a.) In use, when a caregiver presses down on the device 300 (as indicated by the arrow “c”), the foot elements 316 and 326 may press down, such as on the panel 310, and the pressure is distributed to a larger area than would otherwise be applied by foot elements 316 and 318 alone. The panel 310 may be referred to as a “pressure-spreading” element.

Unlike the previous embodiment (300), in this embodiment 300 the panel 310 is part of the device 300, and is also affixed to the bandage 352. Therefore, no detents, pins or the like need to be provided to help properly locate the foot elements 216 and 226 on the panel 210. Rather, this is done “automatically” as described in greater detail hereinbelow.

Recall that the panel 310 is hinged (attached) to the foot 316. However, it may be noticed, in FIG. 3B, that the foot 326 is not on the top (as viewed) surface of the panel 310.

FIG. 3C illustrates that the leg 322′ (prime) may angle slightly inward (towards the opposite leg 312, which will have the effect of locating the foot 326 a little closer to the opposite foot 316. The other right-side leg 324 (not visible in this view) would also angle inward. As the panel 310 swings open (arrow “e”), the legs 322 and 324 and the foot 326 can deflect outward, as indicated by the arrow “f”, resiliently springing back into place so that the (right) foot 326 is positioned atop (on a top surface of the right edge of) the panel 310, for effective transfer of force (arrow “d”) to the wound.

FIG. 3D illustrates an alternative to angling the leg(s) 322 (and 324) inward, to cause the foot 326 to rest upon the panel 310. As shown in FIG. 3D, an inside surface of the foot 326 may be provided with a region of increased thickness, and/or a pawl 330 extending from the foot 326′ (prime) towards the foot 316 so that the (right) foot 326 is positioned atop (on a top surface of the right edge of the) the panel 310, for effective transfer of force (arrow “d”) to the wound.

In a manner similar to that of the previous embodiment (300), a feature of this embodiment is that the handle 300 is configured (sized and shaped) so that a caregiver can insert his hand (fingers of his hand) through the openings 318 and 328 in the left and right sides of the device, securely grasping it, while applying pressure to the wound.

After removing the folded-up bandage 350 from the device 300, the device 300 may be used as a pressure-enhancement member for applying pressure, via the bandage 350, to a wound. Notice (FIG. 3) that the gap 303 is comparable to the gap in the frame member of the pressure enhancement member of the '723 patent. And, in a manner comparable to what was described therein:

During wrapping of the bandage onto a wounded body part, the web of the bandage may be passed though the gap in the frame of the pressure enhancement member into an interior space of the pressure enhancement member. The user then goes back, wrapping the bandage in the opposite direction. The action of wrapping the web in the opposite direction may, according to some embodiments, lock (for example “click in”) panel 310 in a closed position (such as the closed position shown in FIG. 3B).

Wrapping the web in the opposite direction forces the pressure device 300 down (tilting it essentially 90° to the right or to the left) against the dressing such that the pressure enhancement member applies pressure to the wounded area. Panel 310 now extends generally perpendicularly away from the wound area. When device 300 is forced to the right, (right) foot 326 and right side edge 302b apply pressure on the wound while “leg” elements 322 and 324 lie essentially flat on surfaces 352a (indirectly, on dressing 358). The user may continue wrapping the bandage, such that the web covers the tilted device 300, thus applying more pressure on (right) foot 326 and right side edge 302b, which apply pressure on the wound. As opposed to prior art bandages, pressure is being applied essentially along two lines, namely (right) foot 326 and right side edge 302b. This may be advantageous to simultaneously stop a bleeding from two separated ends of a torn blood vessel (such as a vein or an artery).

There have thus been described a number of embodiments of a device which is sized and shaped to function as a holder for packaging the folded-up bandage, and to also function as a handle for applying pressure, via the bandage, to a wound. FIGS. 2A and 2B, 3A and 3B illustrate a device 200, 300, respectively, in conjunction with a “generic” folded bandage 250, 350, respectively, comprising a web and a dressing. A particular bandage-folding configuration which is well-suited to, for example, the devices 200 or 300 will now be described.

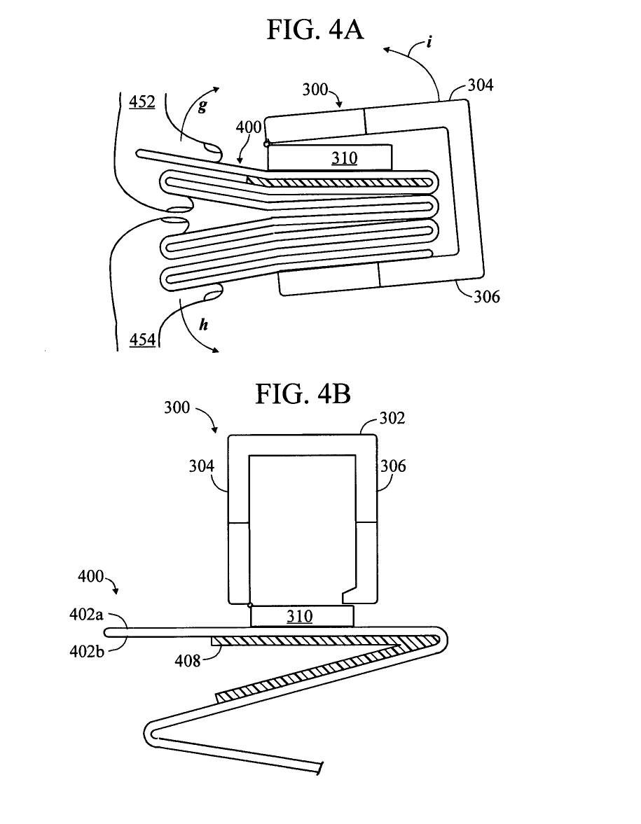

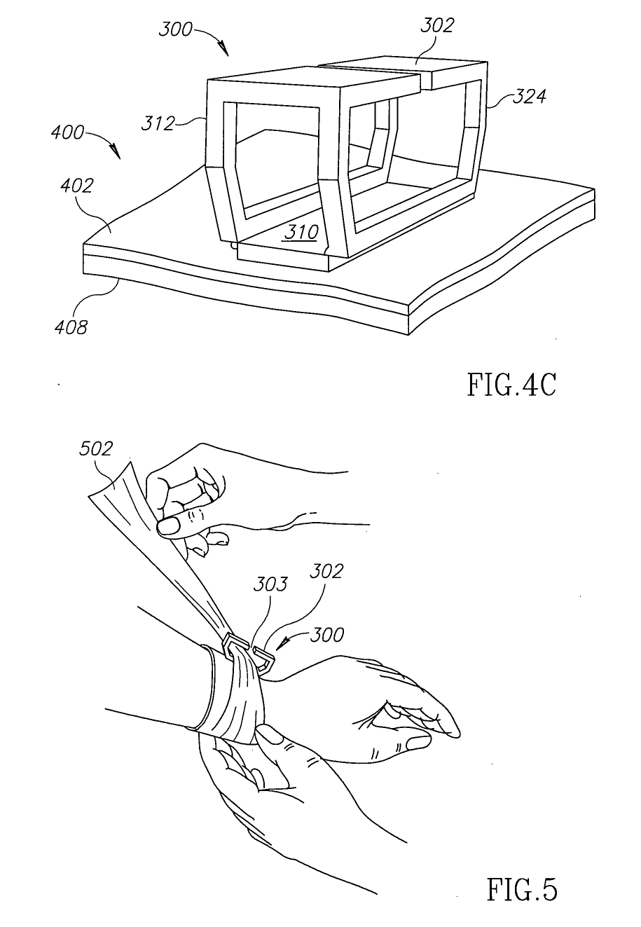

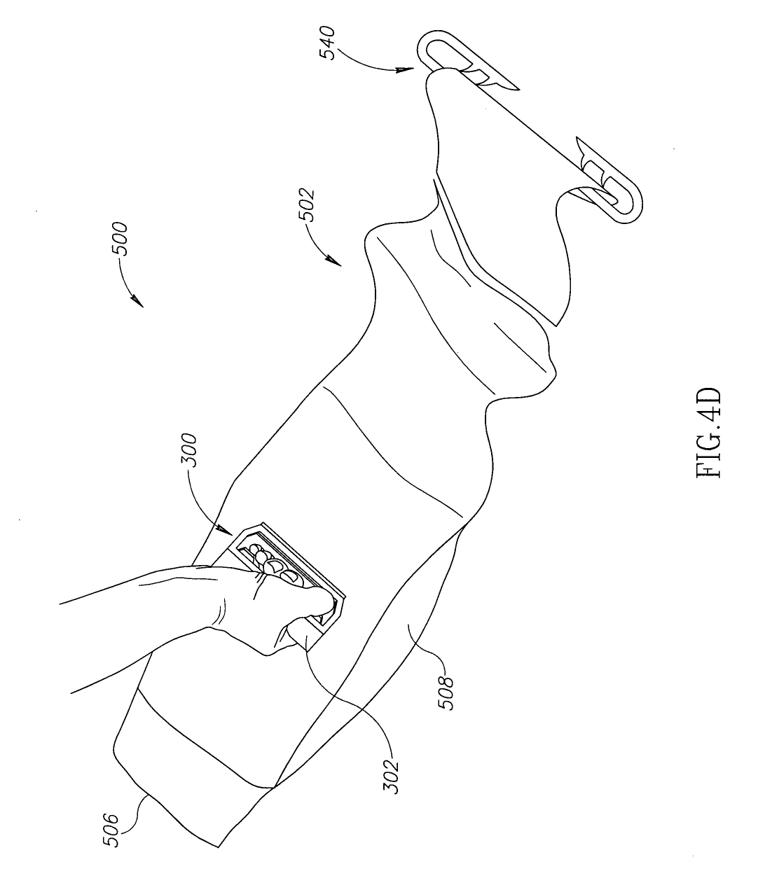

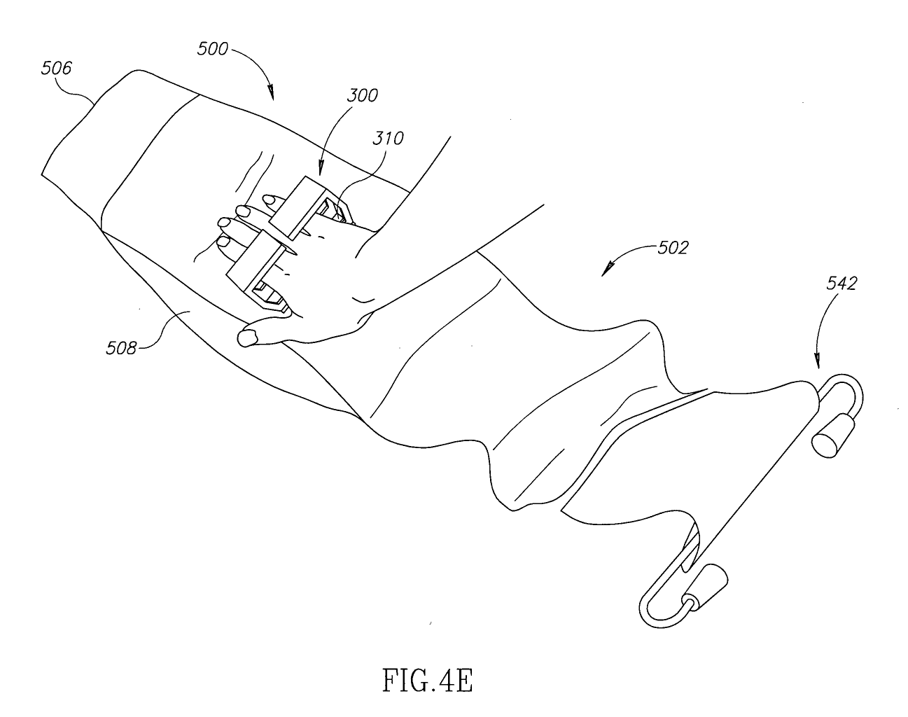

FIG. 4 (similar to FIGS. 2A, 3A) shows a bandage 400, which may be generally similar to the bandage 100 of FIG. 1, in conjunction with a “combination holder & handle” 300 such as the device 300 of FIG. 3.

The bandage 400 comprises an elongated web 402. The web 402 is folded upon itself, such as zig-zag folded, with six (n=6) folds 404a-404f, resulting in seven (n+1) panels 402a-402g. The number of folds may be greater than six (such as seven or eight), or may be less than six.

The panel 402a is an “end panel”. A dressing 408 may be disposed on a “wound” surface of the end panel 402a and an adjacent panel 402b. The dressing 408 may have substantially the same width as the web 402. The dressing 408 may be disposed on the wound surface of the two panels 402a and 402b of the web 402. Thus, when the bandage 400 is folded, the dressing 408 may also consequently be folded, as shown, between the end panel 402a and an adjacent panel 402b.

An additional web portion 406 (compare 106) extends from the end panel 402a. The additional web portion 406 may be grasped by a caregiver when applying the bandage 400 on a wound (not shown). The portion 406 may be approximately the size (length and width) of a panel, such as the panel 402a.

With the bandage 400 in its folded configuration, the additional web portion 406 may be “folded-in”, so that it is disposed within the folded bandage 400, as shown, between the panels 402a and 402b, and within the folded dressing 408.

The panel 310 of the device 300 may be located on the surface of the end panel 402a of the web 402, opposite the dressing 408, and affixed thereto (as indicated by the x′s).

When folded-up, the bandage 400 may have an overall thickness “T”, exclusive of the panel 310. The thickness “T” may, for example, be approximately 3 cm (30 mm). In this example, the overall thickness “T” is approximately equal to seven times the thickness (approximately 4 mm) of each panel (402a-402f), plus twice the thickness of the folded dressing 408, plus a thickness of the additional web portion 406, taking into consideration that the web and dressing are formed of “soft” materials (such as cotton, gauze, elasticized fabric, and the like) which may be compressed when packaged.

FIG. 4 shows the bandage 400 retained by the device 300, which is functioning as a “holder”. Note that only a portion of the bandage 400 is retained within the holder 300, and a remaining (exposed) portion of the bandage 400 is without (outside of) the holder 300.

FIG. 4A illustrates a caregiver (user) grasping the exposed portion of the bandage 400, with two hands 452 and 454, such as a few panels in one hand and a few other panels in another hand, spreading the bandage 400 apart to remove (withdraw) it from the holder 300, as indicated by the arrows “g” and “h”. (Alternatively, the user can pull on the bandage 400 with one hand, and manipulate the holder 300 with the other hand.)

As the bandage 400 is withdrawn from the holder 300, the holder 300 “automatically” moves (pivots) into place, as indicated by the arrow “i”, until it is substantially perpendicular to the web of the bandage, as was shown (for example) in FIG. 4C.

FIG. 4B shows the bandage 400, having been removed from the holder 300, with the bandage 400 unfurling (unfolding), prior to applying the bandage 300 to a wound. A dressing 408 is disposed on the wound surface 402b of the bandage, and the holder 300 is extending substantially perpendicular from the opposite (non-wound) surface 402a of the bandage, opposite the dressing 408.

FIG. 4C shows the bandage 400 with the device 300 in place for applying pressure to a wound. Note that the device 300, functioning as “handle”, is disposed on a portion of the web 402, opposite the dressing 408. (Note that this “version” of the device 300 is with a pawl on the right foot, as in FIG. 4D.)

FIG. 4D shows the bandage 500 having device 300 (as “handle”),an “additional” web portion 506 which extends from the dressing 508 at one end of the web portion 502, a hooking dowel (or “closure bar”) 540 disposed at an opposite end of the web portion 502. The caregiver's hand is shown grasping the top surface 302 of handle 300 to apply immediate pressure to the wound under dressing 508 (not visible), to staunch bleeding.

FIG. 4E shows the bandage 500 having device 300 (as “handle”), an “additional” web portion 506 which extends from the dressing 508 at one end of the web portion 502, a hooking dowel (or “closure bar”) 542 disposed at an opposite end of the web portion 502. The caregiver's hand is shown inside the interior surface of handle 300 applying pressure to bottom surface 310 and thus to the wound under dressing 508 (not visible), to staunch bleeding.

FIG. 5 illustrates the bandage 500 in use. Web 502 having passed over the device 300, more particularly over the top surface 302 of the device 300. The web 502 may then be passed (be urged, or forced) through the gap 303 into the interior space of the device 300. Notice that the web 502 bunches up as it is forced through the gap 303, but the web 502 may be made of a material which allows the web portion 502 to stretch back to nearly full width after being forced through gap 303. Wrapping may continue, as described in the '723 patent, which is incorporated herein by reference in its entirety, such that the direction of wrapping is then reversed, tightly pulling the web back against the device 300, thereby causing (forcing) the device 300 to tilt over, down against the dressing 508 and applying additional pressure to the wounded area. Wrapping may continue, around the device 300, thereby creating a secondary sterile dressing to the entire wound area without additional accessories. Finally, the user may hook the hooking dowels 540 or 542 (as shown for example in FIG. 4D and FIG. 4E respectively) into the wrapped bandage, thereby securing the bandage around the wounded limb.

In a manner similar to that described in the '723 patent, the when bandaging a wound with the bandage 500, the dressing 508 may be placed on a wounded limb (see 460, FIG. 4D), with the device 300 extending away from the wound area, over the wound site. The user may then start to wrap the web portion 502 around the wounded limb.

According to some embodiments, the bandage may further include a retaining device adapted to maintain said elongate wrapping portion in a rolled or folded configuration, allowing a user to gradually unroll or unfold the web, without the remaining elongate wrapping portion unrolling or unfolding itself entirely.

The retaining devices may include an elongated element, a top end element (for example, having a V shape or a ball shape) and a bottom end element (also having for example, a V shape or a ball shape) opposing to top end element, wherein retaining device is adapted to extend through two or more panels of rolled or folded web portion, and releasably retain the web portion in a rolled or folded configuration. The device may be further adapted to allow a user of the bandage to gradually unroll or unfold without removing the device by pulling one or more panels of web portion over top end element, while other panels remain retained. The retaining device may be made of cotton, synthetic fibers, natural fibers, plastic, nylon, silicon or any other appropriate material. More details about potential retaining devices may be found in Israeli Patent Application No. 197854, which is incorporated by reference herein in its entirety.

In the description and claims of the application, each of the words “comprise” “include” and “have”, and forms thereof, are not necessarily limited to members in a list with which the words may be associated.

The invention has been described using various detailed descriptions of embodiments thereof that are provided by way of example and are not intended to limit the scope of the invention. The described embodiments may comprise different features, not all of which are required in all embodiments of the invention. Some embodiments of the invention utilize only some of the features or possible combinations of the features. Variations of embodiments of the invention that are described and embodiments of the invention comprising different combinations of features noted in the described embodiments will occur to persons with skill in the art. It is intended that the scope of the invention be limited only by the claims and that the claims be interpreted to include all such variations and combinations.

1. An apparatus for applying pressure to a wound, the apparatus comprising:
a main body portion having a top panel and two sides, the apparatus is configured to be positioned on a dressing opposing a wound, such that the top panel is essentially parallel to a surface of a dressing.

2. The apparatus of claim 1, wherein the apparatus is configured to be directly or indirectly positioned on the dressing.

3. The apparatus of claim 1, wherein the main body portion is generally U-shaped in cross-section.

4. The apparatus of claim 1, wherein, in use, the apparatus is configured to apply pressure to a wound, by a caregiver pressing against the wound on the top panel of the apparatus.

5. The apparatus of claim 1, wherein the top panel is generally in the form of a rectangle.

6. The apparatus of claim 1, wherein at least one of the two sides comprises an opening of sufficient size that a caregiver can insert fingers of a hand into the openings and apply pressure on the top panel by at least a portion of a palm of the hand.

7. The apparatus of claim 1, wherein:
the top panel comprises four corners;
a first of the two sides comprises:
a first elongated leg element extending from a first of the four corners of the top panel;
a second elongated leg element extending from a second of the four corners of the top panel;
a first elongated foot element extending between bottom ends of the first and the second leg elements; and
a first opening in the first of the two sides which is defined by the first and the second leg elements, the first foot element, and a first side edge of the top panel;
a second of the two sides comprises:
a third elongated leg element extending from a third of the four corners of the top panel;
a fourth elongated leg element extending from a fourth of the four corners of the top panel;
a second elongated foot element extending between bottom ends of the third and the fourth leg elements; and
a second opening in the second of the two sides which is defined by the third and the fourth leg elements, the second foot element, and a second side edge of the top panel.

8. The apparatus of claim 7, wherein the top panel is substantially rectangular.

9. The apparatus of claim 7, wherein the foot elements are substantially parallel with one another.

10. The apparatus of claim 7, wherein the first, second, third and/or fourth elongated leg elements are substantially perpendicular to the top panel.

11. The apparatus of claim 7, wherein the first elongated foot elements extends substantially perpendicularly between bottom ends of the first and the second leg elements; and/or wherein the second elongated foot element extends substantially perpendicularly between bottom ends of the third and the fourth leg elements.

12. The apparatus of claim 7, wherein:
the leg elements extend substantially perpendicularly from an inner surface of the top panel, from the respective four thereof, for a portion of their length, then the leg elements angle inwardly.

13. The apparatus of claim 12, wherein:
the first and the second leg elements angle towards each other; and
the third and the fourth leg elements angle towards each other.

14. The apparatus of claim 7, wherein the foot elements are configured to contact a bottom panel affixed to a bandage, opposite the dressing, wherein, in use, when a caregiver presses down on the top panel, the foot elements press down on the bottom panel, for distributing pressure to a larger area of the wound than would otherwise be applied by the foot elements alone.

15. The apparatus of claim 7, further comprising:
a bottom panel movably attached to the first foot element.

16. The apparatus of claim 15, wherein the bottom panel is movably attached to the first foot element by a living hinge.

17. The apparatus of claim 15, wherein;
in an open position the bottom panel extends up the first side of the apparatus, in an interior space between the two sides of the apparatus; and
in a closed position, the bottom panel extends between the two foot elements at a bottom surface of the apparatus.

18. The apparatus of claim 15, wherein, in use, when a caregiver presses down on the top panel, the foot elements press down on the bottom panel, for distributing pressure to a larger area of the wound than would otherwise be applied by the foot elements alone.

19. The apparatus of claim 15, wherein,
the top panel is generally in the form of a rectangle having a left side edge, a right side edge, a front edge and a back edge, has a width dimension (W) which is a distance between the left side edge and right side edge, and has a length dimension (L) which is a distance between the front edge and the back edge; and
the bottom panel has a length dimension which is at least as great as the length dimension (L) of the apparatus, and has a width dimension which is slightly less than the width (W) of the apparatus.

20. The apparatus of claim 13, wherein:
bottom portions of the third and the fourth leg elements angle slightly inward, so that as the bottom panel swings open, the second foot element deflects outward, resiliently springing back into place so that the second foot is positioned on a top surface of the bottom panel.

21. The apparatus of claim 13, wherein:
an inside surface of the second foot element is provided with a region of increased thickness.

22. The apparatus of claim 1, wherein:
a space between the two sides defines an interior space of the apparatus, and wherein the apparatus is sized and shaped so that a folded-up bandage may be at least partially inserted into the interior space of the apparatus.

23. The apparatus of claim 22, wherein the apparatus is formed of a resilient material, and in use the two sides exert a resilient, retaining force on the folded-up bandage which is inserted into the interior space of the apparatus, and the folded-up bandage may be easily removed from the apparatus.

24. The apparatus of claim 1, wherein the top panel comprises a gap, wherein, in use, during wrapping of a bandage onto a wounded body part, a web of the bandage may be passed though the gap into an interior space which is defined between the two sides of the apparatus.

25. A bandage comprising:
an elongated web, configured to be folded upon itself;
a dressing disposed on a wound surface of the web; and
a pressure enhancement member configured to be directly or indirectly positioned on the dressing opposite the wound, wherein the pressure enhancement member comprises a main body portion having a top panel and two sides, the top panel is essentially parallel to the dressing.

26. The bandage of claim 25, wherein the pressure enhancement member is affixed to the elongated web, opposite the dressing.

27. The bandage of claim 25, wherein the main body portion is generally U-shaped in cross-section.

28. The bandage of claim 25, wherein, in use, the pressure enhancement member is configured to apply pressure to a wound, by a caregiver pressing against the wound on the top panel of the pressure enhancement member.

29. The bandage of claim 25, wherein, in use, the pressure enhancement member is configured to apply pressure to a wound, by a caregiver pressing against the wound on a bottom panel of the pressure enhancement member.

30. The bandage of claim 25, wherein the top panel is generally in the form of a rectangle.

31. The bandage of claim 30, wherein at least one of the two sides comprises an opening of sufficient size that a caregiver can insert fingers of a hand into the openings and apply pressure on the top panel by at least a portion of a palm of the hand.

32-49. (canceled)
